(19) United States
(12) Patent Application Publication
(10) Pub. No.: US 2002/0092534 A1
(43) Pub. Date: Jul. 18, 2002

(54) Cosmetic surgery preview system

(76) Inventor: John M. Shamoun, Newport Beach, CA (US)

Correspondence Address: STRADLING YOCCA CARLSON & RAUTH, IP Department, 660 Newport Center Drive, Suite 1600, P.O. Box 7680, Newport Beach, CA, 92660-6441, US

(21) Appl. No.: 10/039,066

(22) Filed: Jan. 4, 2002

Related U.S. Application Data
(60) Provisional application No. 60/260,510, filed on Jan. 8, 2001.

(51) Int. Cl. A61B 19/00

(52) U.S. Cl. 128/898

(57) ABSTRACT

A method for providing a preview via a network of the potential effects of cosmetic surgery includes selecting a cosmetic surgery procedure, asking at least one question regarding the body of a prospective patient via the network, viewing and/or selecting images related to the body of the patient and/or the selected cosmetic surgery procedure, answering the question(s) via the network and using the answers to form preview image of the prospective patient showing the potential effects of the selected cosmetic surgery procedure.

### PRIORITY CLAIM

This patent application claims the benefit of the filing date of U.S. Provision Patent Application Serial No. 60/260,510, filed on Jan. 8, 2001 and entitled COSMETIC SURGERY PREVIEW SYSTEM, the entire contents of which are hereby expressly incorporated by reference

### FIELD OF THE INVENTION

The present invention relates generally to cosmetic surgery. The present invention relates more particularly to a method for providing a preview via a network (such as the Internet) of the potential effects of cosmetic surgery.

### BACKGROUND OF THE INVENTION

Cosmetic surgery previews for allowing patients to see the potential effects of a cosmetic surgery procedure, either in a doctor's office or via the Internet, are known. However, contemporary cosmetic surgery preview systems require that prospective patients provide pictures of themselves.

Many patients are reluctant to provide pictures of themselves. This is particularly true when the cosmetic surgery procedure involves an intimate part of the perspective patient's body. For example, according to contemporary practice providing a preview of a breast augmentation procedure requires that a picture of the perspective patient's breast be provided.

Providing a picture of an intimate body part is particularly objectionable when the preview is being requested and provided via the Internet. Many perspective patient's are extremely reluctant to transmit a picture of an intimate body part via the Internet, due to privacy concerns. Moreover, the requirement for a picture of the perspective patient or any body part thereof is an undesirable obstacle toward providing the desired preview. Not only must such a picture be taken, but the picture must be transmitted in digital form in order to provide a preview via the Internet. The generation and transmission of such a picture as a digital image generally requires some degree of technical knowledge and skill.

Therefore, it is desirable to provide a method for forming a preview of the potential results of a cosmetic surgery procedure, such as a breast enhancement procedure, via a network such as the Internet, wherein the perspective patients are not required to provide pictures of themselves.

### SUMMARY OF THE INVENTION

The present invention specifically addresses and alleviates the above-mentioned deficiencies associated with the prior art. More particularly, the present invention comprises a method for providing a preview via a network, such as the Internet, of the potential effects of cosmetic surgery, such as a breast augmentation procedure. The method comprises selecting a cosmetic surgery procedure, asking at least one question, preferably a series of questions, regarding the body of a patient via the network, answering the question(s) via the network, and then using the answers to form a preview image of the patient showing the potential effects of the selected cosmetic surgery procedure.

Optionally, photographs, drawings, diagrams, or other images may be utilized to clarify the questions, as discussed in detail below.

Optionally, the patient may select from a series of photographs, drawings, diagrams, or other images, the image which most closely approximates their body, some portion of their body, or some characteristic of their body.

These, as well as other advantages of the present invention, will be more apparent from the following description and drawings. It is understood that changes in the specific structure shown and described may be made within the scope of the claims without departing from the spirit of the invention.

### BRIEF DESCRIPTION OF THE DRAWINGS

FIG. 1 is a front view of a woman' breasts, showing some exemplary measurements for use in the present invention; and

FIG. 2 is a side view of one breast, showing an additional measurement for use according to the present invention.

### DETAILED DESCRIPTION OF THE INVENTION

The detailed description set forth below in connection with the appended drawings is intended as a description of the presently preferred embodiment of the invention, and is not intended to represent the only form in which the present invention may be constructed or utilized. The detailed description sets forth the construction and functions of the invention, as well as the sequence of steps for operating the invention in connection with the illustrated embodiment. It is to be understood, however, that the same or equivalent functions may be accomplished by different embodiments that are also intended to be encompassed within the spirit and scope of the invention.

The present invention comprises a method for providing a preview via a network (such as the Internet) of the potential affects of cosmetic surgery, such as a breast augmentation procedure. The method comprises selecting a cosmetic surgery procedure, then asking at least one question regarding the body of a patient via the network, then answering the question(s) via the network, and then using the answers to form a preview of the patient showing the potential effects of the selected cosmetic surgery procedure.

Preferably, a series of questions regarding the body of the patent is asked, so as to facilitate generation of the preview of the patent showing the potential effects of the selected cosmetic surgery procedure. As those skilled in the art will appreciate, the exact number of questions and the nature of the questions will depend upon the body parts for which cosmetic surgery is desired and upon the nature of the cosmetic surgery procedure desired.

According to the preferred embodiment of the present invention, one or more photographs, drawings, diagrams, or other images may be utilized to clarify any or all of the questions. Such images may be utilized to provide examples of the anatomical characteristics relevant to the questions. Such images may be utilized to provide examples of body parts having various characteristics. Such images may be utilized to show the results of cosmetic surgery procedures.

Photographs, drawings, diagrams, or other images of a body, part of a body, or characteristic of a body may be utilized such that a patient selects the image which most closely matches the patients own body, part of the body, or characteristic of the body. In this manner, more accurate and reliable answers to the questions are likely to be provided.

As mentioned above, the network may comprise the Internet. Alternatively, the network may comprise any other desired network, such as a local area network (LAN) or a wide area network (WAN).

Selecting a cosmetic surgery procedure preferably comprises selecting a cosmetic surgery procedure such as a cosmetic surgery procedure for the breasts, a cosmetic surgery procedure for the eyes, a cosmetic surgery procedure for the removal of fat, a cosmetic surgery procedure for the lips, a cosmetic surgery procedure for the ears, a cosmetic surgery procedure for the face, a cosmetic surgery procedure for the hair, a cosmetic surgery procedure for the mitigation of scar tissue, a cosmetic surgery procedure for the nose, a cosmetic surgery procedure for the chin, and a cosmetic surgery procedure for the cheeks.

Preferably, a specific cosmetic surgery procedure within the specified category is selected. For example, the cosmetic surgery procedure may comprise selecting one of a plurality of different breast enhancement procedures.

Asking at least one question preferably comprises asking a plurality of questions, such as questions about the present form, e.g., shape, size, coloration, of the body or of a part of the body of a prospective patient.

Further, asking at least one question preferably comprises asking at least one question about the history of the body of the patient, such as whether or not the patient has had a child.

According to the preferred embodiment of the present invention, a plurality of multiple choice questions are asked. As discussed above, photographs, drawings, diagrams, or other images may be utilized in combination with these questions so as to facilitate their being answered by a patient in an accurate manner. When relating to breast augmentation procedures, examples of such questions and some multiple choice answers preferably include:

1. How would you describe your breasts?

I have never been pregnant and I have little, if any breast tissue.

I have very nice, small or medium sized B cup breasts with essentially no droopiness.

I have never been pregnant and have little or any breast tissue.

I have very nice C cup breasts with excellent shape and symmetry.

I had B cup breasts prior to pregnancy; at the time of pregnancy, I went to a C or D, and now I have shrunken, small A-B cup breasts with missing volume and not much droopiness.

I had C cup breasts until childbirth, at which time they became D cups; now I have large B cup to C cup breasts which appear droopy.

2. Age?

3. Weight and height?

4. Breast size: A cup, B cup, C cup, or D cup?

5. I have significant asymmetry? Yes or No.

6. My ancestral background is:

Asian.

Mid Eastern.

Afro-American.

American.

Hispanic.

7. My body type is:

Mesomorphic (muscular).

ectomorphic (very thin).

endomorphic (large boned).

8. I would describe my chest wall (if I cut my body in half horizontally and looked at it on cross-section) as:

barrel chest.

thin pencil chest.

normal cylinder.

abnormal because it is sunken in at the middle.

abnormal because it protrudes in the middle.

9. I would describe my breast tissue as:

dense (hard to the touch).

fatty (very soft to the touch).

mixed.

10. I would describe my breast shape as:

round.

tubular.

flat with no existing breast tissue.

breast tissue only at the level of the nipple or above, with no breast tissue below the nipple.

perfect teardrop.

11. I would describe my skin as:

stretchy with stretch marks; very droopy.

thick, virgin type skin with excellent pliability.

12. I have had more than one pregnancy and have breast feed more than one child:

yes.

no.

13. My weight, with the exception of pregnancy, was within 10-20 lbs. of normal.

yes.

no.

14. I would describe my areolae as:

dilated.

stretched and enlarged.

normal dimensions.

normal diameter.

very small diameter (less than 3.5 cm).

15. I would describe the pigment of my areola as:

darkly pigmented.

lightly pigmented.

16. I would describe my nipple projection as:

large.

normal.

inverted.

17. Pre-existing history of breast cancer in:

sister.

maternal relative.

one relative, two relatives, greater than two relatives.

18. I prefer:

a very natural look to my breasts, (conservative.).

for relatives or friends to not notice much of an enlargement in clothing, however, when wearing skimpy clothes or a bathing suit, more noticeable image changes are apparent;

19. In addition, I desire:

a very natural appearing breast when nude.

an obvious difference in clothing as well as nude and in all clothing wear.

a very fake looking breast with headlight appearance and round, full volume (very unnatural look nude).

obvious breast implant look.

Preferably the questions comprise questions regarding a plurality of measurement results. The questions also preferably comprise questions regarding the development of the relevant anatomical structure and any other anatomical perimeters or characteristics necessary to develop a preview image of the desired cosmetic surgery procedure. For example, for a breast augmentation procedure the following measurements may be requested: sternal notch to nipple distance, bilateral inframammary crease to nipple distance, nipple to nipple distance, and/or bilateral base dimension.

Preferably, a plurality of multiple choice questions (optionally with accompanying photographs, diagrams or other images) are asked, a plurality of measurement results (optionally with line drawings or other images to clarify precisely how the measurements are to be made) are requested and/or a plurality of photographs or images are selected from. The results for the the above-asked questions, measurements and/or photograph/image selection are used to form a preview image of the patient showing the likely or potential effects of the selected cosmetic surgery procedure. Thus, the answers to such questions are used, at least in part, to form the preview image.

Optionally, the preview image is a pre-existing image which most closely matches a predicted outcome of the cosmetic surgery procedure. Alternatively, a nonpre-existing image or a newly generated image may be utilized. In any event, an image is utilized which attempts to closely match a predicted outcome of the cosmetic surgery procedure.

The present invention thus mitigates a need for a prospective patient to provide a picture of himself or herself in order to preview the results of a desired cosmetic surgery procedure. As those skilled in the art will appreciate, many patients are reluctant to provide such a picture, particularly via a non-secure communication system such as the Internet.

Thus, according to one aspect of the present invention, a client computer facilitates selection of a cosmetic surgery procedure and a host computer communicates at least one question, preferably a plurality of questions, regarding the body or body part of the patient via the network to the client computer. The client computer also facilitates answering of the question(s) via the network. A computer, which may optionally be the host computer, uses the answers to form a preview image of the patient showing the potential effect of the selected cosmetic surgery procedure. It is worthwhile to note that the host computer which communicates the question(s) and photographs or other images to the client computer and the computer which uses the answers to form the preview image may either be the same computer or may be two different computers, as desired.

According to the present invention, a question database facilitates communication of at least one question regarding the body of the patient via the network to the client computer. A processor is preferably configured to use answers to the question(s) to form a preview image of the patient showing a potential outcome of a cosmetic surgery procedure.

The method of the present invention may be used for a wide variety of different cosmetic procedures, wherein the aesthetic value of the outcome of the procedure is likely to be important to the patient. Another example of a cosmetic procedure which may be practiced according to the present invention is a cosmetic dentistry procedure.

According to the cosmetic dentistry procedure application of the present invention, at least one question regarding the teeth of a patient is asked via the network. The question(s) are then answered by the prospective patient, preferably utilizing the same network. As those skilled in the art will appreciate, either the same or another network (with respect to the network via which the questions were asked) may be utilized to answer any of the questions according to the present invention. The answers are used to form a preview image of the patient, showing the potential outcome of the selected cosmetic dentistry procedure.

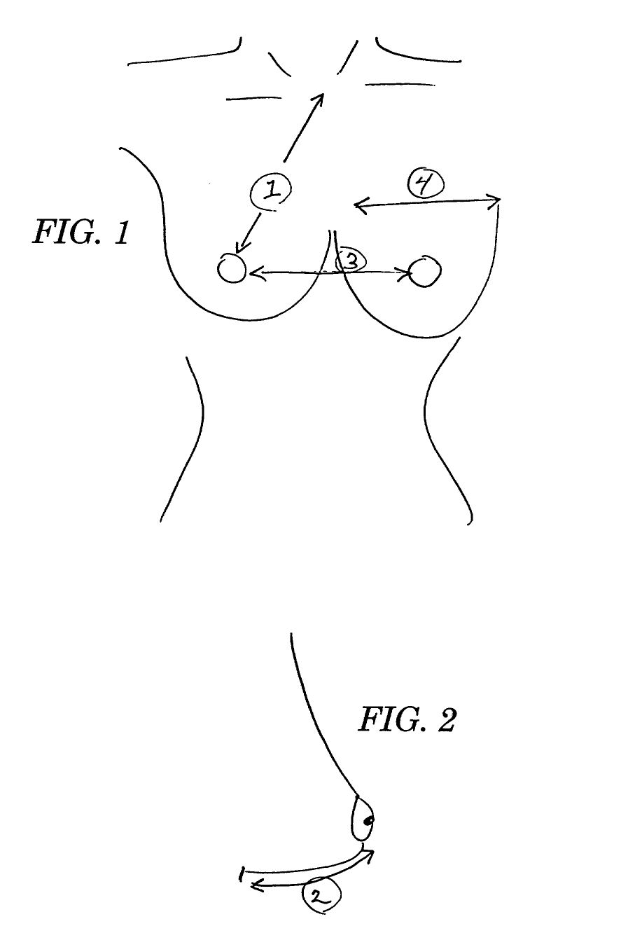

Referring now to FIGS. 1 and 2, some exemplary measurements used in the practice of the present invention for forming a preview image for breast augmentation is provided. The sternal notch to nipple distance is shown as dimension 1 (FIG. 1). The bilateral inframammary crease to nipple distance is shown as dimension 2 (FIG. 2). The nipple to nipple distance is shown as dimension 3 (FIG. 3). The bilateral base dimension is shown as dimension 4 (FIG. 4).

As those skilled in the art will appreciate, various other dimensions and/or anatomical characterizations are likewise suitable for use in breast augmentation surgeries, and/or other medical procedures.

The following represents examples of how the answers to the questions will aid in effecting the preview image. The answers help determine the breast volume, breast tissue/skin relationships and the skin quality. Pregnancy, size, and shape of breasts determine these qualities and will influence how the preview image looks.

Some answers relate to the age of the patient, as well as the height and weight thereof. This will influence the preview image in that the younger the patient, the better quality skin and the more dense the breast tissue. This will also influence the preview image. The weight and height of the patient determine the base dimensions of a particular implant, and influence the preview image.

Breast cup size also influences the preview image in terms of the volume of tissue present and its relationship to the chest diameter.

Asymmetry can also influence the preview image in that certain characteristics of the discrepancy in size will definitely determine what a particular breast can potentially look like post-operatively.

The ancestral background question relating to the particular ethnicity of a patient determines the color of the preview image and facilitates closely matching the pre-operative skin tone.

The body type, as to whether the patient is muscular, very thin, or large boned, determines the nature of the exact preview image utilized to best depict the body morphology.

The chest wall shape determines the preview image which most closely matches a pre-operative image. This chest wall shape influences the particular outcome of any breast augmentation procedure due to the bony characteristics of each individual woman's chest.

The chest wall itself serves as a platform for the implant to rest upon and will influence the positioning of any particular implant, and therefore the post-operative result.

The volume of tissue is important. Further, very dense, hard tissue will not conform to the underlying implant as well as very soft, butter-like tissues. Therefore, the image visualized must closely match the preview image based on questions regarding volume, density and firmness.

At least one question preferably relates to the specific shape of a breast which will definitely influence the particular shape post-operatively. Some tubular breast deformities require skin excision, and the amount of skin excision depends on the particular shape.

The particular skin type determines the quality of the skin which will influence the amount of post-operative droopiness present when any implant is utilized. It is important to utilize a preview image that most closely approximates the skin quality of any individual in order to closely approximate the post-operative result.

Some answers relate to pregnancy and the parenchymal tissue and skin characteristics related to the same. It is precisely the skin characteristics and parenchymal changes that will influence the breast shape, and therefore this question is considered importance.

Some questions relate to the characteristics of nipple areolar complex on an individual breast mound. It is the nipple areolar complex characteristics that determine the fine tuning of each preview image in order to most closely approximate the post-operative image.

Answers which relating to breast cancer risk will not influence the preview image whatsoever. However, it will impact upon the decision-making process concerning the particular incision utilized as well as the approach as to whether the implants are under or over the muscle. Such questions may optionally be omitted.

Some questions relate to an individual woman's preference as to what she would like for her breasts to look like. Some women prefer a very natural looking breast. By way of contrast, other women prefer a very obvious breast implant look.

According to the present invention, a perspective patient selects a desired cosmetic surgery procedure, such as a breast augmentation procedure, and is provided with a preview image which attempts to show the perspective patient what the effects of the selected cosmetic procedure would be for that patient.

The preview image is formed by asking the perspective patient a series of questions which are intended to elicit information regarding the particular relevant anatomic features of the patient. This information is then used to either form or select an image which is appropriate for that particular patient. Either one of a plurality of pre-stored images may be selected, so as to reflect the likely results of the cosmetic surgery procedure for that particular patient, or a new image is formed using the elicited information.

Thus, a database of pre-stored post cosmetic surgery images may be formed. A particular image is selected from the database based upon answers to the questions provided by the perspective patient. For example, information relating to the present size, shape, and coloration of the perspective patient's breasts may be utilized to define a group of images within the database which are likely to closely approximate the desired results of a selected breast augmentation surgery procedure. Further questions regarding anatomical details of the perspective patient narrow the scope of the images within the database which are likely to provide a substantially accurate preview image. Additionally, questions regarding the history of the patient (such as whether the patient has ever given birth), questions regarding the precise type of operation to be performed (whether a breast implant is to be inserted behind or in front of the muscle) and questions regarding the desired outcome (whether a natural look or an artificial look is desired) may optionally be asked to better define the preview image.

Alternatively, a computer generated image may be formed based upon the answers to the questions provided by the perspective patient.

In either instance (whether the post-cosmetic surgery images are retrieved from a database or are generated anew), the answers to the questions provided by the perspective patient define the preview image and eliminate the need for the perspective patient to provide a picture of himself or herself.

In this manner, a perspective patient may view preview images showing the potential results of various different cosmetic surgery procedures. Seeing the potential results of various different cosmetic surgery procedures may help the patient to decide which, if any, cosmetic surgery procedure the perspective patient desires.

Optionally, a particular cosmetic surgery procedure may be suggested to the perspective patient, based upon the questions answered by the perspective patient. In some instances, answers to the questions will tend to indicate that some cosmetic surgery procedures are more suitable for a particular patient than other cosmetic surgery procedures. Indeed, some procedures may be ruled out altogether. Thus, the present invention facilitates a determination of which cosmetic surgery procedure is best suited for a particular individual.

Optionally, answers to the most frequently asked questions regarding the specific cosmetic surgery procedure which had been determined to be most suitable for the perspective patient will also be provided. If appropriate, information regarding various different alternatives which are considered to be suitable for the perspective patient may also be provided. Any other information deemed to be of interest to the prospective patient may be provided, including advertisements, doctor's names and phone numbers, and links to web pages or Internet discussion or newsgroups.

Optionally, a plurality of images showing the potential outcomes of different cosmetic surgery procedures may be provided simultaneously, so as to facilitate a side-by-side comparison which may help the perspective patient choose the particular procedure to be performed. For example, a perspective breast enhancement patient may desire to see a side-by-side comparison of how both a behind the muscle and an in front of the muscle implant procedure will look for her.

The present invention further comprises a method for providing a preview via a network of the potential effects of cosmetic surgery, the method comprising: selecting a cosmetic surgery procedure; asking at least one question regarding the body of a patient via the network; answering at least one question regarding the body of a patient via the network; and using the answers to form a preview image of the patient showing the potential effects of the selected cosmetic surgery procedure.

The present invention further comprises a method for providing a preview via a network of the potential effects of cosmetic surgery, the method comprising: providing a selection of cosmetic surgery procedures; asking at least one question regarding the body of a patient via the network; receiving answers the question(s) via the network; and using the answers to form a preview image of the patient showing the potential effects of the selected cosmetic surgery procedure.

The present invention further comprises system for providing a preview via a network of the potential effects of cosmetic surgery, the system comprising: a client computer for facilitating selection of a cosmetic surgery procedure; a host computer for communicating at least one question regarding the body of a patient via the network to the client computer; the client computer also facilitating answering the question(s) via the network; and a computer configured to use the answers to form a preview image of the patient showing the potential effects of the selected cosmetic surgery procedure.

The computer configured to use the answers is preferably the host computer.

The present invention further comprises a host computer for providing a preview via a network of the potential effects of cosmetic surgery, the host computer comprising: a question database for facilitating communication of at least one question regarding the body of a patient via the network to the client computer; and a processor configured to use answers to the question(s) to form a preview image of the patient showing a potential outcome of a cosmetic surgery procedure.

The present invention further comprises a method stored on a computer readable media, the method providing a preview via a network of the potential effects of cosmetic surgery, the method comprising: selecting a cosmetic surgery procedure; asking at least one question regarding the body of a patient via the network; answering the question(s) via the network; and using the answers to form a preview image of the patient showing the potential effects of the selected cosmetic surgery procedure.

The present invention further comprises a data structure comprising: a list of questions to ask a person so as to facilitate forming a preview of the potential effects of cosmetic surgery.

The present invention further comprises a method for providing a preview via a network of the potential effects of cosmetic surgery, the method comprising the steps of: a selecting step for selecting a cosmetic surgery procedure; a query step for asking at least one question regarding the body of a patient via the network; a response step for answering the question(s) via the network; and a processing step for using the answers to form a preview image of the patient showing the potential effects of the selected cosmetic surgery procedure.

The present invention further comprises a system for providing a preview via a network of the potential effects of cosmetic surgery, the system comprising: first computer means for facilitating selection of a cosmetic surgery procedure; second computer means for communicating at least one question regarding the body of a patient via the network to the client computer; the first computer also facilitating answering the question(s) via the network; and computer means for using the answers to form a preview image of the patient showing the potential effects of the selected cosmetic surgery procedure.

The present invention further comprises a method for providing a preview via a network of the potential effects of cosmetic dentistry, the method comprising: selecting a cosmetic dentistry procedure; asking at least one question regarding the teeth of a patient via the network; answering the question(s) via the network; and using the answers to form a preview image of the patient showing the potential effects of the selected cosmetic dentistry procedure.

According to the preferred embodiment of the present invention, a combination of answers to questions, measurements, and selected images are used to enhance the quality and accuracy of a preview image provided for a patient, so as to show the patient the likely outcome of a cosmetic surgery procedure.

It is understood that the exemplary method for providing a preview via a network of the potential effects of cosmetic surgery described herein and shown in the drawings represents only presently preferred embodiments of the invention. Indeed various modifications and additions may be made to such embodiments without departing from the spirit and scope of the invention. For example, the present invention may be utilized to show the outcome of various different types of procedures including non-cosmetic procedures and non-medical procedures. Further, the present invention may be practiced via a plurality of different types of communications systems other than data networks such as the Internet. For example, the present invention may be practiced via telephone systems, facsimile systems, written media such as the U.S. mail, and via any other desired communication method. Further, the preview image may be displayed upon a computer screen, printed material, or via any other desired means. Thus, these and other modifications and additions may be obvious to those skilled in the art and may be implemented to adapt the present invention for use in a variety of different applications.

What is claimed is:

1. A method for providing a preview via a network of the potential effects of cosmetic surgery, the method comprising:
selecting a cosmetic surgery procedure;
asking at least one question regarding the body of a patient via the network;
answering the question(s) via the network; and
using the answers to form a preview image of the patient showing the potential effects of the selected cosmetic surgery procedure.

2. The method as recited in claim 1, wherein at least one image is provided in association with the question(s) so as to facilitate answering of the question(s).

3. The method as recited in claim 1, further comprising selecting at least one image which matches some characteristic of a patient.

4. The method as recited in claim 1, wherein selecting a cosmetic surgery procedure comprises selecting a cosmetic surgery category from the group consisting of:
a cosmetic surgery procedure for the breasts;
a cosmetic surgery procedure for the eyes;
a cosmetic surgery procedure for the removal of fat;
a cosmetic surgery procedure for the lips;
a cosmetic surgery procedure for the ears;
a cosmetic surgery procedure for the face;
a cosmetic surgery procedure for the hair;
a cosmetic surgery procedure for the mitigation of scar tissue;
a cosmetic surgery procedure for the nose;
a cosmetic surgery procedure for the chin; and
a cosmetic surgery procedure for the cheeks.

5. The method as recited in claim 4, wherein selecting a cosmetic surgery procedure comprises selecting a specific cosmetic surgery procedure.

6. The method as recited in claim 4, wherein selecting a cosmetic surgery procedure comprises selecting a specific cosmetic surgery procedure within the selected category.

7. The method as recited in claim 1, wherein selecting a cosmetic surgery procedure comprises selecting one of a plurality of different breast enhancement procedures.

8. The method as recited in claim 1, wherein asking at least one question comprises asking a plurality of questions about the present form, e.g., shapes, size, coloration, of body of the patient.

9. The method as recited in claim 1, wherein asking at least one question comprises asking at least one question about the history of the body of the patient.

10. The method as recited in claim 1, wherein asking at least one question comprises asking a plurality of multiple choice questions.

11. The method as recited in claim 1, wherein asking at least one question comprises asking at least some of the following questions:
A) How would you describe your breasts?
I have never been pregnant and I have little, if any breast tissue;
I have very nice, small or medium sized B cup breasts with essentially no droopiness;
I have never been pregnant and have little or any breast tissue;
I have very nice C cup breasts with excellent shape and symmetry;
I had B cup breasts prior to pregnancy; at the time of pregnancy, I went to a C or D, and now I have shrunken, small A-B cup breasts with missing volume and not much droopiness;
I had C cup breasts until childbirth, at which time they became D cups; now I have large B cup to C cup breasts which appear droopy;
B) Age?;
C) Weight and height?;
D) Breast size: A cup, B cup, C cup, or D cup?;
E) I have significant asymmetry;
F) My ancestral background is:
Asian;
Mid Eastern;
Afro-American;
American;
Hispanic;
G) My body type is:
Mesomorphic (muscular);
ectomorphic (very thin);
endomorphic (large boned);
H) I would describe my chest wall (if I cut my body in half and looked at it on cross-section) as:
barrel chest;
thin pencil chest;
normal cylinder;
abnormal because it is sunken in at the middle;
abnormal because it protrudes in the middle;
I) I would describe my breast tissue as:
dense (hard to the touch);
fatty (very soft to the touch); mixed;
J) I would describe my breast shape as:
round;
tubular;
flat with no existing breast tissue, or breast tissue only at the level of the nipple or above, with no breast tissue below the nipple;
perfect teardrop;
K) I would describe my skin as:
stretchy with stretch marks; very droopy;
thick, virgin type skin with excellent pliability;
L) I have had more than one pregnancy and have breast feed more than one child:
yes;
no;
M) My weight with the exception of pregnancy was within 10-20lbs. of normal;
N) I would describe my areolae as:
dilated; stretched and enlarged; normal dimensions;
normal diameter, very small diameter (less than 3.5 cm);
O) I would-describe the pigment of my areola as:
darkly pigmented;
lightly pigmented;
P) I would describe my nipple projection as:
large;
normal;
inverted;
Q) Preexisting history of breast cancer in:
sister;
maternal relative;
one relative, two relatives, greater than two relatives;
R) I prefer:
a very natural look to my breasts, (conservative);
for relatives or friends to not notice much of an enlargement in clothing; however, when wearing skimpy clothes or a bathing suit, more noticeable image changes are apparent;
S) In addition, I desire:
a very natural appearing breast when nude;
an obvious difference in clothing as well as nude and in all clothing wear;
a very fake looking breast with headlight appearance and round, full volume (very unnatural look nude); and
obvious breast implant look.

12. The method as recited in claim 1, wherein asking at least one question comprises asking for a plurality of measurement results.

13. The method as recited in claim 1, wherein asking at least one question comprises asking for the following measurement results:
sternal notch to nipple distance;
bilateral inframammary crease to nipple distance;
nipple to nipple distance; and
bilateral base dimension.

14. The method as recited in claim 1, wherein asking at least one question comprises asking a plurality of multiple choice questions and asking for a plurality of measurement results.

15. The method as recited in claim 1, wherein using the answers to from a preview image of the patient showing the potential effects of the selected cosmetic surgery procedure comprises using the answers to form a preview image of the affected body part.

16. The method as recited in claim 1, wherein using the answers to from a preview image of the patient showing the potential effects of the selected cosmetic surgery procedure comprises selecting a pre-existing image which most closely matches a predicted outcome of the cosmetic surgery procedure.

17. The method as recited in claim 1, wherein using the answers to from a preview image of the patient showing the potential effects of the selected cosmetic surgery procedure comprises generating a new image which most closely matches a predicted outcome of the cosmetic surgery procedure.

18. A method for showing potential results of a breast enhancement cosmetic surgery procedure via the Internet without requiring a picture of the prospective patient, the method comprising:
selecting a breast enhancement cosmetic surgery procedure to be performed;
A) How would you describe your breasts?
I have never been pregnant and I have little, if any breast tissue;
I have very nice, small or medium sized B cup breasts with essentially no droopiness;
I have been pregnant and have little or any breast tissue;
I have very nice C cup breasts with excellent shape and symmetry;
I had B cup breasts prior to pregnancy; at the time of pregnancy, I went to a C or D, and now l have shrunken, small A-B cup breasts with missing volume and not much droopiness;
I had C cup breasts until childbirth, at which time they became D cups; now I have large B cup to C cup breasts which appear droopy;
B) Age?
C) Weight and height?
D) Breast size: A cup, B cup, C cup, or D cup?;
E) I have significant asymmetry;
F) My ancestral background is:
Asian;
Mid Eastern;
Afro-American;
American;
Hispanic;
G) My body type is:
Mesomorphic (muscular);
ectomorphic (very thin);
endomorphic (large boned);
H) I would describe my chest wall (if I cut my body in half and looked at it on cross-section) as:
barrel chest;
thin pencil chest;
normal cylinder;
abnormal because it is sunken in at the middle;
abnormal because it protrudes in the middle;
I) I would describe my breast tissue as:
dense (hard to the touch);
fatty (very soft to the touch)
mixed;
J) I would describe my breast shape as:
round;
tubular;
flat with no existing breast tissue, or breast tissue only at the level of the nipple or above, with no breast tissue below the nipple;
perfect teardrop;
K) I would describe my skin as:
stretchy with stretch marks;
very droopy;
thick, virgin type skin with excellent pliability;
L) I have had more than one pregnancy and have breast feed more than one child:
yes;
no;
M) My weight with the exception of pregnancy was within 10-20 lbs. of normal;
N) I would describe my areolae as:
dilated;
stretched and enlarged;
normal dimensions;
normal diameter, very small diameter (less than 3.5 cm);
O) I would describe the pigment of my areola as:
darkly pigmented;
lightly pigmented;
P) I would describe my nipple projection as:
large;
normal;
inverted;
Q) Preexisting history of breast cancer in:
sister;
maternal relative;
one relative, two relatives, greater than two relatives;
R) I prefer:
a very natural look to my breasts, (conservative);
for relatives or friends to not notice much of an enlargement in clothing; however, when wearing skimpy clothes or a bathing suit, more noticeable image changes are apparent;
S) In addition, I desire:
a very natural appearing breast when nude;
an obvious difference in clothing as well as nude and in all clothing wear;
a very fake looking breast with headlight appearance and round, full volume (very unnatural look nude); and
obvious breast implant look.

19. A method for providing a preview via a network of the potential effects of cosmetic surgery, the method comprising:
providing a selection of cosmetic surgery procedures;
asking at least one question regarding the body of a patient via the network;
receiving answers the question(s) via the network; and
using the answers to form a preview image of the patient showing the potential effects of the selected cosmetic surgery procedure.

20. A method for providing a preview via a network of the potential effects of cosmetic surgery, the method comprising:
selecting a cosmetic surgery procedure;
asking at least one question regarding the body of a patient via the network work;
selecting at least one image which matches an anatomical characteristic of a patient via the network; and
using the selected image(s) to form a preview image of the patient showing the potential effects of the selected cosmetic surgery procedure.
